SEQUENCE LISTING (4 sequences)

SEQ ID NO: 1 - DNA, 26 bp, Homo sapiens
  source 1..26: mol_type = genomic DNA
gcctgaggtagtaggttgtatagtta

SEQ ID NO: 2 - RNA, 22 nt, Homo sapiens
  source 1..22: mol_type = mRNA
aagttctgttatacactcaggc

SEQ ID NO: 3 - RNA, 22 nt, Homo sapiens
  source 1..22: mol_type = mRNA
tgagaactgaattccatgggtt

SEQ ID NO: 4 - DNA, 27 bp, Homo sapiens
  source 1..27: mol_type = genomic DNA
ggtacctattgtgttgagtaacggtga

(19) United States
(12) Patent Application Publication
(10) Pub. No.: US 2023/0190849 A1
(43) Pub. Date: Jun. 22, 2023

(54) USE OF HUANGQI EXTRACT FOR SUPPRESSING SARS-COV-2 ENTRY AND TREATING COVID-19 RELATED CYTOKINE STORM

(71) Applicant: PhytoHealth Corporation, Taipei (TW)

(72) Inventor: I-Lin LEE, Taipei (TW)

(21) Appl. No.: 17/957,288

(22) Filed: Sep. 30, 2022

Related U.S. Application Data
(60) Provisional application No. 63/251,210, filed on Oct. 1, 2021.

(51) Int. Cl. A61K 36/481; A61P 31/14; A61P 37/06

(52) U.S. Cl. CPC A61K 36/481; A61P 31/14; A61P 37/06

(57) ABSTRACT

The extract of Huangqi (Astragalus membranaceus) is a drug or composition with multiple effects. It is not only acting as an inhibitor of viral infection of host cells, but also has the emerging potential to inhibit viral replication and cytokine storm by mediating miRNA expression.

### CROSS-REFERENCE TO RELATED APPLICATIONS

This application is priority under 35 U.S.C. § 119(e) to U.S. Provisional Patent Application No. 63/251,210, filed on Oct. 1, 2021, which is incorporated by reference in its entirety herein.

### REFERENCE TO AN ELECTRONIC SEQUENCE LISTING

The contents of the electronic sequence listing (SEQUENCE.xml; Size: 4821 bytes; and Date of Creation: Dec. 27, 2022) is herein incorporated by reference in its entirety.

### FIELD OF THE INVENTION

This disclosure relates to a use of an extract of Huangqi (Astragalus membranaceus) to prevent, treat or relieving SARS-Cov-2 infection and preventing cytokine storm.

### BACKGROUND OF THE INVENTION

The rapid spread of SARS-CoV-2 has started the pandemic outbreak since late 2019, then has been making a huge impact on public health systems worldwide. The mortality rate of COVID-19 ranges from 0.7 to 4%, and the transmission rate is increasing due to increased lethal SARS-CoV-2 variants. Discovering new therapeutic drug is urgently needed to prevent medical supports overload, though a number of registered anti-viral clinical trials are ongoing. Unfortunately, most known mechanisms of anti-viral agents targeted at limited pathways, which may deviate the pathophysiology of SARS-CoV-2 infection or may cause intolerable adverse effects. Conventional antiviral chemical drugs or vaccine development are limited to costly and time-consuming for monitoring safety issues, as well as their ambiguous effects on variant strains, which are unmet pandemic prevention needs. Concerning the safety of an anti-viral agent, traditional Chinese medicine (TCM) may be an excellent candidate since it only exerts minimal toxicities and mild side-effects. There are also numerous complex TCM formulae in testing the therapeutic effects toward COVID-19, but most of them are lack of rationales based on molecular mechanisms.

According to current progresses in developing drugs to treat COVID-19, scientists focused on two different phases during the course of infection: antiviral drugs for viral phase, and anti-inflammatory/immunomodulatory drugs for inflammatory phase. Among the potential anti-viral targets screen, spike protein, angiotensin converting enzyme 2 (ACE2), transmembrane serine protease 2 (TMPRSS2), protease, endosome, and RNA dependent RNA replication (RdRp) are research hot spots in progress. Other researchers addressed on ARDS, which is a consequence of virus-induced uncontrolled systemic cytokine release syndrome. Most COVID-19 patients with ARDS are characterized by elevated various cytokine levels, including interleukin (IL)-2, IL-6, IL-7, interferon-γ inducible protein 10 (CXCL10), granulocyte colony-stimulating factor (G-CSF), and tumor necrosis factor-α (TNF-α).

We identified one common Chinese herb, the extract of Astragalus membranaceus, which showed similar anti-SARS-CoV-2 characteristics. Next, we validated the anti-SARS-CoV-2 effects of this TCM's using the normal bronchial epithelium cell line model to mimic the therapeutic effects in treating the COVID-19 patients.

Huangqi (Astragalus membranaceus) is an anti-viral immunomodulatory compound rich in Astragalus polysaccharide (APS), thereby possibly being used as an ideal solution to treat SARS-CoV-2 infection in our predication. However, the functional ingredients of Huangqi responsible for inducing innate let-7a, miR-148, or exerting anti-viral effects remain poorly understood.

Moreover, we also aimed at discovering how Huangqi act on alleviating ARDS. Previous clinical data revealed that IL-6 and TNF-α were the most critically cytokines secreted in severe COVID-19 patients. The secretion and activation of IL-6 are mainly caused by TNF-α and Toll like receptors (TLRs). Nuclear factor kappa B (NF-κB) generates several inflammatory protein transcriptions, such as TNF-α. In addition, the interaction between NF-κB and IL-6 forms a positive feedback loop, which contributes to deadly cytokine release syndrome.

An extract of Astragalus membranaceus (PhytoHealth Corporation, Taiwan, ROC), an injectable, contains a mixture of polysaccharides extracted, isolated and purified from the Astragalus membranaceus, a kind of Traditional Chinese Medicine (TCM) used for treating “qi deficiency (lack of energy)”.

Furthermore, an extract of Astragalus membranaceus has stimulated hematopoiesis effects, it can promote the proliferation and maturation of bone marrow and spleen precursor cells in mice treated with 5-fluorouracil or mitomycin C, and promote the recovery of peripheral blood leukocytes, erythrocytes, and platelets in sublethally irradiated mice model.

Due to rapid evolution and high mutation rate of SARS-CoV-2, multiple target-mapping strategy may be considered to increase effectiveness in treating emerging SARS-CoV-2 variant infection. It should be noted that asymptomatic patients accounting for 40% to 45% of SARS-CoV-2 infections could transmit virus for an extended period longer than 14 days. Severe COVID-19 patients have been reported to suffer cytokine release syndrome related symptoms, which can lead to the fatal ARDS with an incident rate of 41.8%. In addition, survival patients from ARDS still had high risk of disease sequel since lungs were not able to regenerate by itself, or in worse cases, ARDS disease might develop to pulmonary fibrosis. As the complicated situation of rapid viral variant mutation and risky after-effect, there is an urgent demanding of the drugs which have the ability to suppress a broad spectrum of targets in the SARS-CoV-2 related mechanisms

Accordingly, it is very important to develop an effective, safe and inhibiting SARS-CoV-2 entry and COVID-19 related cytokine storm pharmaceutical composition.

### SUMMARY OF THE INVENTION

In present invention, it is found that an extract of Astragalus membranaceus for the patient suffering from the SARS-Cov-2 infection, the extract of Astragalus membranaceus can suppress the binding of the SARS-CoV-2 viral spike protein to the human ACE2 receptor, elevating the miR-146a, the let-7a or the miR-148b expression to perform its normal cellular function, so it can effectively prevent, treat or relieving the SARS-Cov-2 infection and preventing the cytokine storm.

In view of the above-mentioned problem, the present invention provides a method for preventing the SARS-Cov-2 infection in a subject, comprising administering to the subject an extract of Astragalus membranaceus, wherein the extract of Astragalus membranaceus can suppress the binding of the SARS-CoV-2 viral spike protein to the human ACE2 receptor.

In some embodiments, the extract of Astragalus membranaceus is selected from the group of the extract of Astragalus membranaceus A, B and C (hereinafter referred to as: EAM A, EAM B, EAM C).

In some embodiments, the EAM A is the extract of Astragalus membranaceus containing 55%-80% of total sugar content, the EAM B is the extract Astragalus membranaceus containing 25%-50% of total sugar content, the EAM C is the extract of Astragalus membranaceus containing 85%-100% of total sugar content.

In some embodiments, the EAM C is comprising an arabinose:galactose ratio ranging from about 5% to about 15% arabinose; less than about 1.5% rhamnose; from about 3% to about 7% galactose; less than about 4% galacturonic acid; and, from about 70% to about 90% glucose.

Further, the present invention also provides a method for treating or relieving the SARS-Cov-2 infection in a subject, comprising administering to the subject an extract of Astragalus membranaceus, wherein the extract of Astragalus membranaceus targeting a miR-146a, a let-7a or a miR-148b, elevating the miR-146a, the let-7a and the miR-148b expression to perform its normal cellular function.

In some embodiments, the extract of Astragalus membranaceus can reduce the SARS-CoV2 M^{pro} activity IC₅₀ concentration.

Further, the present invention also provides a method for preventing the cytokine storm caused by the SARS-CoV-2 infection in a subject, comprising administering to the subject an extract of Astragalus membranaceus, wherein the extract of Astragalus membranaceus targeting a miR-146a, a let-7a or a miR-148b, elevating the miR-146a, the let-7a or the miR-148b expression to perform its normal cellular function.

In some embodiments, the extract of Astragalus membranaceus can reduce the IL-6, the IL-10 or the TNF-α expression.

Further, the present invention also provides a method for preventing the cytokine storm in a subject, comprising administering to the subject an extract of Astragalus membranaceus, wherein the extract of Astragalus membranaceus targeting a miR-146a, a let-7a or a miR-148b, elevating the miR-146a, the let-7a or the miR-148b expression to perform its normal cellular function.

It is to be understood that both the foregoing general description and the following detailed description are exemplary and explanatory only and are not restrictive of the invention.

### BRIEF DESCRIPTION OF THE DRAWINGS

FIGS. 1A-1C show the results of the induction of the let-7a, the miR-148b, and the miR-146a levels by different concentration of the extract of Astragalus membranaceus A, B, C (EAM A, EAM B, EAM C).

FIGS. 2A-2E show the results of inhibition of the cytokine release level by different concentration of the extract of Astragalus membranaceus A, B, C (EAM A, EAM B, EAM C).

FIGS. 3A-3B show that the EAM B is the potential inhibitors of the SARS-CoV2 protease.

FIGS. 4A-4B show the effect of the extract of Astragalus membranaceus B, A (EAM B, EAM A) on the binding of SARS-CoV2-spike with ACE2 and syncytia formation.

FIGS. 5A, 5B, 6A and 6B show the effect of the extract of Astragalus membranaceus C (EAM C) on the binding of SARS-CoV2-spike with ACE2 and syncytia formation.

FIGS. 7A-7E show the reduction of binding efficacy of the wild-type, alpha, delta, and gamma of the trimeric spike protein binding to the ACE2 reaching statistical analysis by different concentration of the extract of Astragalus membranaceus A, B (EAM A, EAM B).

FIGS. 8A-8E show the Variant specificity of the extract of Astragalus membranaceus C (EAM C) against trimeric spike protein binding to ACE2.

### DETAILED DESCRIPTION OF THE INVENTION

The “EAM A” in the present invention refers to the extract of Astragalus membranaceus A containing 55%-80%% of total sugar content.

The “EAM B” in the present invention refers to the extract of Astragalus membranaceus B containing 25%-50%% of total sugar content.

The “EAM C” in the present invention refers to the extract of Astragalus membranaceus C containing 85%-100% of total sugar content.

The EAM C is comprising an arabinose:galactose ratio ranging from about 5% to about 15% arabinose; less than about 1.5% rhamnose; from about 3% to about 7% galactose; less than about 4% galacturonic acid; and, from about 70% to about 90% glucose.

The present invention provides a method for preventing the SARS-Cov-2 infection in a subject, comprising administering to the subject an extract of Astragalus membranaceus, wherein the extract can suppress the binding of the SARS-CoV-2 viral spike protein to the human ACE2 receptor.

Further, the present invention also provides a method for treating or relieving the SARS-Cov-2 infection in a subject, comprising administering to the subject an extract of Astragalus membranaceus wherein the extract targeting a miR-146a, a let-7a or a miR-148b, elevating the miR-146a, the let-7a or the miR-148b expression to perform its normal cellular function.

Further, the present invention also provides a method for preventing the cytokine storm caused by the SARS-CoV-2 infection in a subject, comprising administering to the subject an extract of Astragalus membranaceus, wherein the extract targeting a miR-146a, a let-7a or a miR-148b, elevating the miR-146a, the let-7a or the miR-148b expression to perform its normal cellular function.

Further, the present invention also provides a method for preventing the cytokine storm in a subject, comprising administering to the subject an extract of Astragalus membranaceus, wherein the extract targeting a miR-146a, a let-7a or a miR-148b, elevating the miR-146a, the let-7a or the miR-148b expression to perform its normal cellular function.

In some embodiments, the effective concentration of the extract of Astragalus membranaceus is 10˜8000 μg/ml.

In some embodiments, the extract of Astragalus membranaceus is administered by injection, infusion, intravenous, or oral.

In some embodiments, the extract of Astragalus membranaceus can be tablet, pill, granule, powder, capsule, or liquid.

Additional specific embodiments of the present invention include, but are not limited to the following:

### Example 1

Preparation of the Extract of Astragalus membranaceus
(a) Put the astragalus chips in a container A, add a pure water to form a solution A;
(b) Heat the solution A formed in step (a) at a time in 2-4 hours, the temperature is 80-100° C.;
(c) Collect and concentrate the solution of step (b) to obtain an extraction liquid A;
(d) Put the extraction liquid A of step (c) in a container B, add 30˜50% ethanol, stir and precipitate, collect and concentrate the supernatant to obtain a concentrated solution A, the concentrated solution A is EAM A;
(e) Add the concentrated solution A obtained in step (d) in 75%-95% ethanol, stir and precipitate, collect the precipitate B;
(f) Dehydrate and filter the precipitate B of step (e), dissolve and precipitate with ethanol, centrifuge the precipitate to obtain a product B, the product B is EAM B; and
(g) Concentrate, refine, and filter the product B of step (f) to obtain the product C, the product C is EAM C.

### Example 2

miRNA Measurement Assay

We conducted the initial feasibility study using BEAS2B cell line model. BEAS2B cells derived from normal bronchial epithelium of non-cancerous human were used for screening biological agents affecting respiratory tract infection mechanisms. To determine whether candidate drugs can induce let-7a or miR-148 expression, cells (1×10⁶) were seeded in the 10 cm dish 24 h before drug treatment. Samples were collected after 24 h of treatment. TRIzol® reagent was used for extraction, and samples were stored at −80° C. The miRNA let-7a and miR-148 expression levels were determined by real-time qPCR using U54 as internal control.

The primer sequences are as follows:

Hsa-let-7a PCR primer sequence:
(SEQ ID NO: 1)
5’-GCCTGAGGTAGTAGGTTGTATAGTTA-3’.
Hsa-miR-148b PCR primer sequence:
(SEQ ID NO: 2)
5’-AAGUUCUGUUAUACACUCAGGC-3’.
Has-miR-146a PCR primer sequence:
(SEQ ID NO: 3)
5’-UGAGAACUGAAUUCCAUGGGUU-3’.
The U54 (homo) PCR primer seq:
(SEQ ID NO: 4)
5’-GGTACCTATTGTGTTGAGTAACGGTGA-3’.

The experiments were performed by Phalanx miRNAOneArray® Profiling provided by Phalanx Biotech Group.

The EtOH and H₂O Extractions of Huangqi Elevated Let-7a, miR-148b, and miR-146a Expression

Since elevating the expression of let-7a, miR-148b, and miR-146a was suggested to be beneficial for COVID-19 treatment, we established the experiment to study the effects of candidate drugs on turning on these targeted miRNAs. To determine the safe doses, BEAS2B cells were treated with candidate TCM in EtOH- or H₂O-extractions for 24 h in 96-well plates. SRB assay was used to evaluate the cell viability.

After 24 h of treatment with candidate TCM, total cellular RNA was extracted using TRIzol reagent. The miRNA let-7a, miR-148b, and miR-146a expression level were measured by RT-qPCR followed the protocol by Phalanx miRNAOneArray® Profiling (Phalanx Biotech Group).

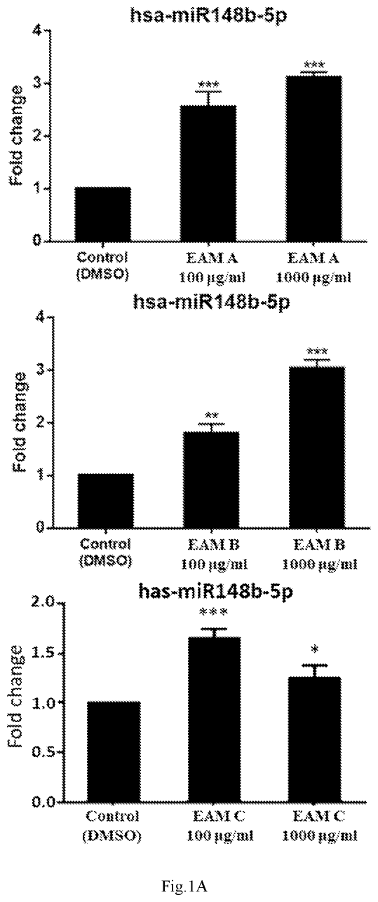

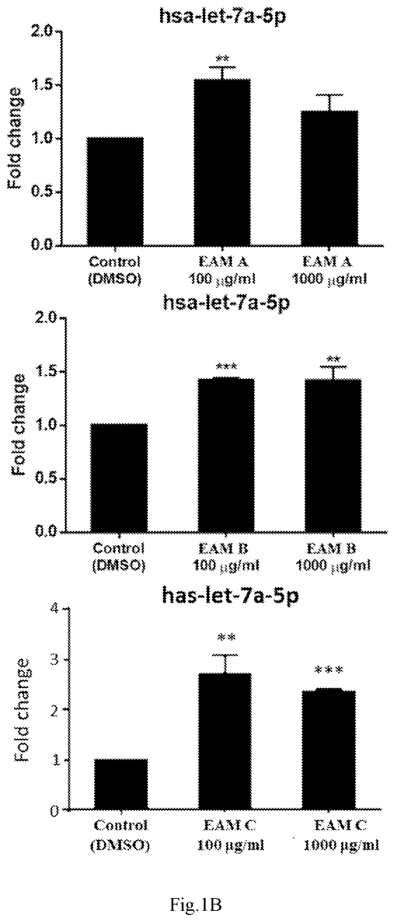

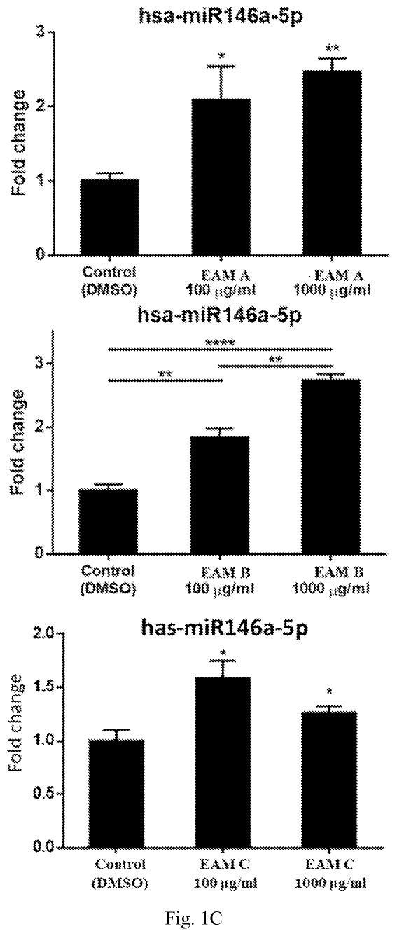

In contrast, all treatment of EAM B could noticeably enhance the manifesting level of targeted miRNAs. Both EAM A and EAM B could effectively increase let-7a amount from 1.2 to 1.5 times (FIG. 1A), while they were more capable of up-regulating miR-148b and miR-146a expression from approximately 2 to 3 times (FIG. 1B-1C).

These results suggested that EAM B could effectively increase the expression of let-7a, miR-148b, and miR-146a, contributing to viral replication blocking possibility. Meanwhile, miR-148b has been proved to not only regulate the expression of HLA-C to interfere viral antigen presenting process to immune system, but also involve in generating inflammatory responses in various diseases. Besides, miR-148 down-regulates NF-κB pathway by means of targeting and inhibiting the expression of MyD88 which can receive immune signaling from outside of the cells, thereby considered as a positive target for defeating SARS-CoV-2. Moreover, miR-146a, known to have a role in the inflammatory regulation by altering macrophages, and NF-κB, is a valid candidate marker for ARDS. The consistent decretion of miR-146a-5p level was observed in COVID-19 patients, and linked to the severity of COVID-19 related inflammation. We had predicted and demonstrated that EAM B could induce the level of miR-146a, which might benefit for COVID-19 treatment (FIG. 1C)

EAM C Elevated Let-7a, miR-148b, and miR-146a Expression

After 24 h of treatment with candidate TCM, total cellular RNA was extracted using TRIzol reagent. The miRNA let-7a and miR-148b expression level were measured by real-time qPCR followed the protocol by Phalanx miRNAOneArray® Profiling (Phalanx Biotech Group). U54 was used as the internal control.

The results showed that EAM C showed its efficiency in enhancing the expression of all three targeted miRNAs by both doses but the impact of low dose was slightly more prominent than that of high dose. In this part, EAM C 100 and 1000 μg/ml respectively augmented significantly let-7a, miR-148b, and miR-146a expression for 2.7 and 2.4 folds, 1.6 and 1.2 folds, and 1.6 and 1.3 folds (FIG. 1A-1C). These results suggested that EAM C could effectively increase the expression of let-7a, miR-148b, and miR-146a.

### Example 3

Validation Cytokine Storm Inhibiting Ability on Immune Cells of Candidate TCM

After 6 or 24 h of treatment with potential drugs, cell medium was collected to quantify cytokine release level by ELISA. The treatment of stimulator LPS alone was used as the control, by which we detected the secretion level of IL-6 and TNF-α which are two of the mostly cytokines presenting in COVID-19 patients' plasma.

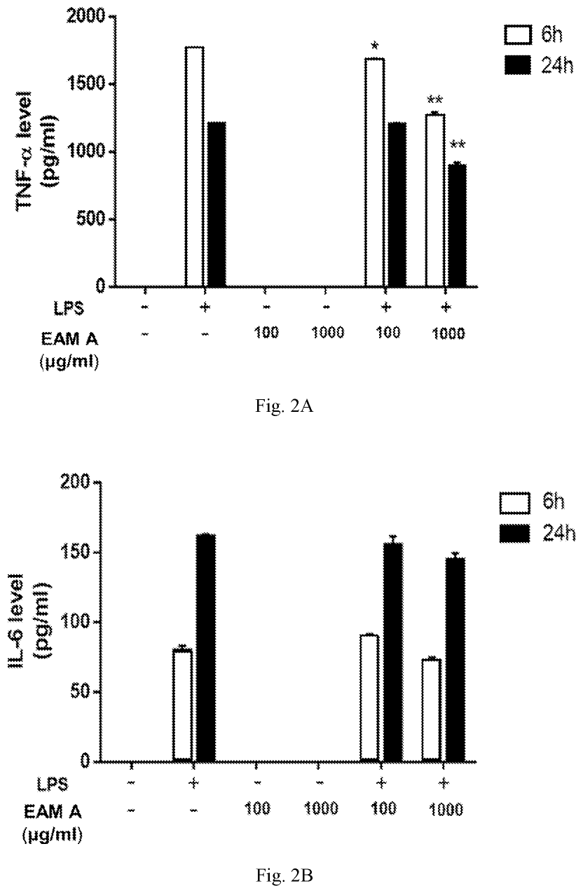

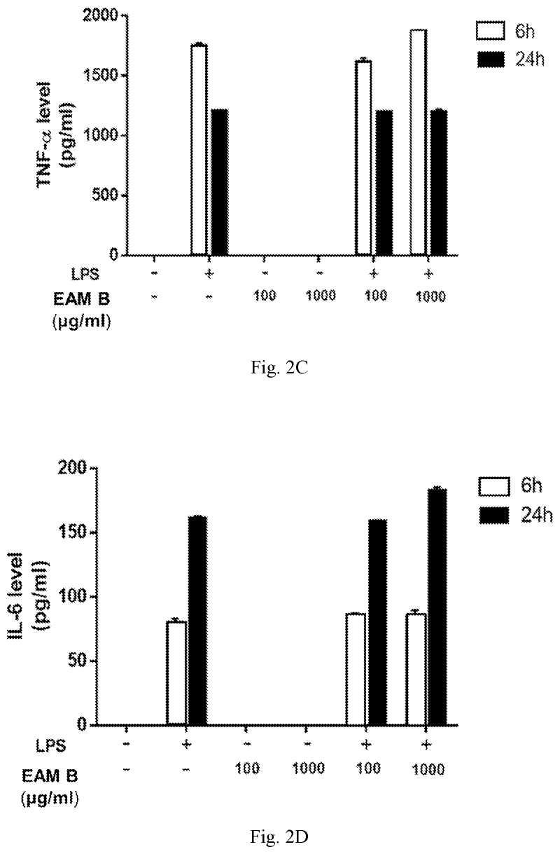

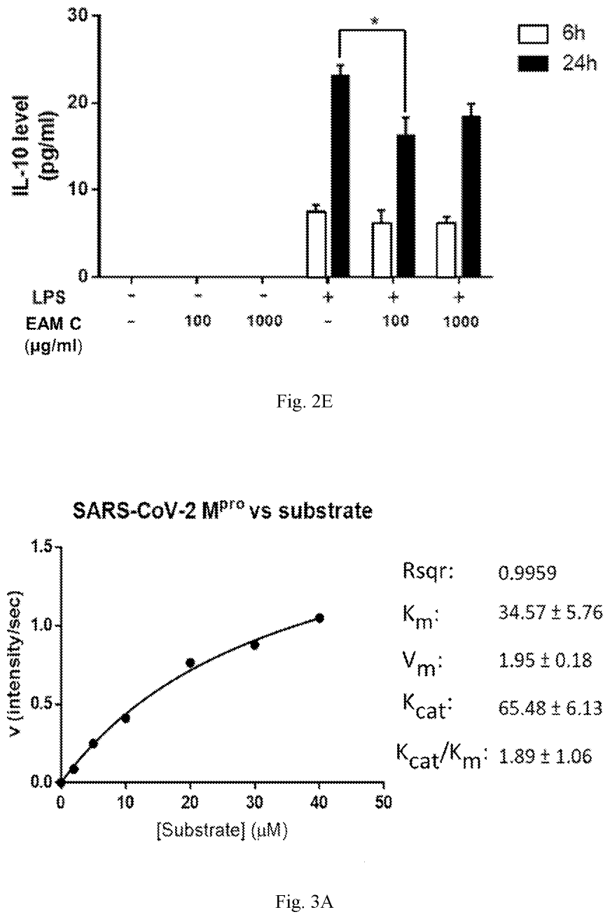

Remarkably, EAM A could inhibit TNF-α. In inflammation environment, both EAM A in 100 and 1000 μg/ml were capable of suppressing this cytokine in a time and dose dependent manner. However, the higher dose showed better effect with the noticeable inhibition at both timepoints, while lower dose could only slightly down-regulate TNF-α secretion level (FIG. 2)

Because LPS is a potent immune stimulus that causes cytokine storm, LPS stimulation was used as a model to investigate the capability of Huangqi treatments in the inhibition of cytokine productions. In the inflammatory environment, EAM A at 100 and 1,000 μg/ml could suppress TNF-α in a time- and dose-dependent manner. While the higher dose of EAM A could cause a noticeable inhibition of TNF-α release at both time points, the lower dose could only lower the secretion level of TNF-α slightly. These data suggested that these drug candidates were able to inhibit cytokine storms by reducing the release of IL-6 or TNF-α, which were abundant in acute-phase COVID-19 patients.

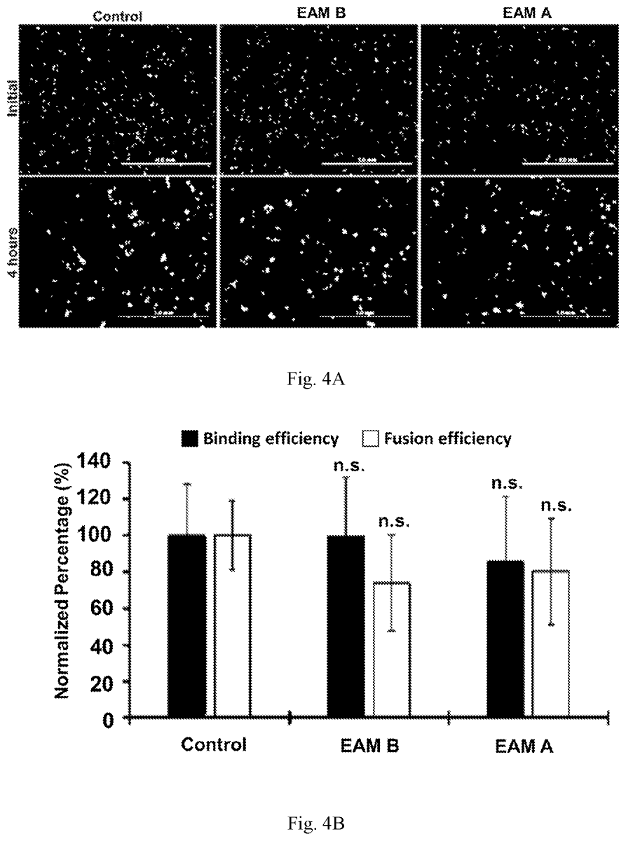

Taken together, these extracts had high chances of inhibiting cytokine storm, indicated by the reduction of IL-6 or TNF-α expression, which are highly presented in acute-phase COVID-19 patients, after the treatment. We intersected all the significant targets among Huangqi, let-7a, and miR-148b. To clarify whether our candidate TCM could treat cytokine storm, we designed in vitro LPS-induced THP-1 cell assay to determine cytokine level via ELISA. Even though, not all the cytokines were reduced via our TCMs showed in supplementary FIG. 4, the Huangqi respectively inhibited TNF-α or IL-6 that strength fully complemented their function on anti-SARS-CoV-2 (FIG. 2A) Especially, LDC extract of APC was more benefit than DC formulate on cytokine storm interference. Therefore, we preferred to design the combination therapy for Huangqi owing their auxiliary function.

The Cytokine Release Level with Candidate TCM Treatment.

After 6 or 24 h of treatment, THP-1 cell medium was collected and performed ELISA kit to measure cytokine release amount. Statistical analysis was carried out with t-test. Unfortunately, some of cytokines did not significantly change via TCMs effect, the different herbal reduced TNF-α and IL-6 level to improve the combination therapy. Compared EAM B data, EAM A was more efficacy on anti-SARS-CoV-2 owing to the latter decreases TNF-α which played a critical role in the initiative cytokine storm. (FIG. 2A-2D)

### Example 4

Cytokine Determination Assays. Inhibition of LPS-Induced IL-10 Release Levels by EAM C

THP-1 cell line was used. It was differentiated by PMA (50 ng/ml) treatment for 24 h. LPS 100 ng/ml was used as the stimulator to mimic the inflammatory condition and considered as the control. The potential drugs were added and incubated at 37° C. for 6 or 24 h. The cell medium was collected and stored at −20° C. Levels of cytokines induced by treated cells were determined by ELISA assay. The supernatants were analyzed on Nunc™MaxiSorp™ 96-well plates separately for cytokines using human uncoated ELISA kit following manufacturer's protocol (Invitrogen, Thermofisher). OD value was measure by OD reader Infinite 200Pro using Tecani-control program at the wavelength of 450 nm and 570 nm.

Result

Validation Cytokine Storm Inhibiting Ability on Immune Cells of Candidate TCM

After 6 or 24 h of treatment with potential drugs, cell medium was collected to quantify cytokine release level by ELISA. The treatment of stimulator LPS alone was used as the control, by which we detected the secretion level of IL-10, which is highly presented in COVID-19 patients' plasma.

EAM C, on the other hand, could suppress only IL-10. Without the presence of LPS, EAM C alone could not induce IL-10 on differentiate THP-1 cells. With the presence of LPS, EAM C 100 μg/ml significantly suppressed IL-10 release amount at timepoint 24 h, while the cytokine amount at timepoint 6 h was slightly inhibited by both doses of EAM C (FIG. 2E). This Astragalus-based herbal 4medicine demonstrated its faculty of suppressing the cytokines which highly contributed to cytokine storm in COVID-19 patients, implying their effect on reducing ARDS which may be a result of cytokine release syndrome.

With the presence of LPS, EAM C 100 μg/ml significantly suppressed IL-10 release amount at time point 24 h, while the cytokine amount at time point 6 h was slightly inhibited by both doses of EAM C (FIG. 2E). This Astragalus-based herbal medicine demonstrated its faculty of suppressing the cytokines which highly contributed to cytokine storm in COVID-19 patients.

### Example 5

Inhibition of M^{pro} Activity and Determination of the Half Maximal Inhibitory Concentration (IC₅₀)

For determining inhibitory ability of M^{pro} activity, EAM B was used as the inhibitor which was incubated at 30° C. for 3 min together with fluorogenic peptide substrate in PBS, followed by protease addition and equilibrated at 30° C. for 3 min. The IC₅₀ value was obtained by following equation:

$$v = \frac{{v}_{0}}{( 1 + {IC}_{50}^{n} ) {/ [ I ]}^{n}}$$

in which v is the velocity at different concentration of the incubated inhibitor [I] and v₀ is the initial velocity without inhibitor, whereas n is the Hill constant.

The Cell-Cell Fusion Assay

Calu-3 were first seeded in 12-well plate (1×10⁶ per well) to form a single-layer of cell films as target cells. BHK-21 cells (4×10⁵ per well) were transfected with both EGFP and Spike plasmids (the original Wuhan strain) in a ratio of 1:5 for 24 h, harvested using Cell Dissociation Buffer (Gibco) and resuspended in serum free DMEM (Gibco). EGFP/Spike-coexpressing BHK-21 cells that were used as donor cells were incubated on a single-layer of Calu-3 cell film for cell-cell contact at 4° C. for 45 minutes prior to PBS washing and growth medium replacing to removing unbound cells. After 4° C. incubation, the temperature was switched to 37° C. and cells were incubated for additional 4 h. Five fields of GFP-expressing cell images were randomly acquired by an inverted fluorescence microscope (Olympus IX70) at time points, 0 and 4 h, respectively. To quantify binding efficiency of GFP-positive BHK-21 cells with Calu-3 cells in both control and TCM-treated groups, the initial numbers of GFP-positive BHK-21 cells attached with Calu-3 cells were counted. The number of GFP cells in control groups was defined as 100% of the binding efficiency. The effects of TCM on the bind efficiency were determined by the percentage of the binding efficiency normalized by control. To quantify the formation of syncytium form, the extension area of GFP-expressing cell images was quantified by ImageJ software. The fold change of GFP area in control groups from 0 to 4 h was delimited to 100% of the fusion efficiency. The effects of TCM on the syncytium formation were calculated according the following equation:

$$Normalized percentage ( \% ) = \frac{the fold change of GFP area in TCM - treated group}{the fold change of GFP area in control group} \times 100$$

Result

Suppression of M^{pro} Protease Activity of SARS-CoV-2 Implicated a Potential Therapeutic Role of Huangqi in COVID-19

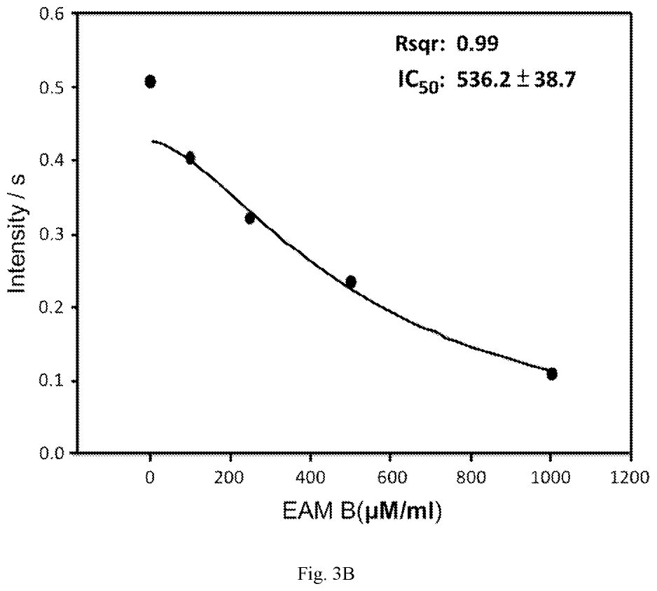

In order to examine the repression of SARS-CoV-2 M^{pro} by EAM B we established an assay in which viral M^{pro} activity could be measured by the means of fluorescence occurring when M^{pro} proteolyzed at cleavage site of fluorogenic peptide substrate. As a result, the max reaction velocity Vₘ was 1.95±0.18 (intensity/sec), Michaelis constant Kₘ was 34.57±5.76 mol/L, turnover number K_{cat} was 65.48±6.13 (1/sec), K_{cat}/Kₘ was 1.89±1.06. The coefficient of determination (Rsqr) in this regression model is 0.9959 (FIG. 3A). To evaluate the ability to inhibit SARS-CoV-2 M^{pro} protease, Huangqi extract EAM B were used as the inhibitors in protease activity assay. This drug evidently showed the capacity of suppressing M^{pro} activity and reached IC₅₀ at 536.21±38.74 μg/ml, respectively (FIG. 3B), indicating that this drug can suppress SARS-CoV-2 M^{pro} protease.

Further, we designed a protease activity assay to evaluate whether our candidate TCM could interrupt SARS-CoV-2 replication through suppression of M^{pro} protease activity of SARS-CoV-2. The enzyme activity of SARS-CoV-2 M^{pro} has been validated in FIG. 3A. Using this in vitro analysis, we disclosed that EAM B could restrain M^{pro} activity of SARS-CoV-2 in a dose dependent manner (FIG. 3B), implying this herb may suppress SARS-CoV-2 replication in infected patients. Moreover, their results failed to show anti-SARS-CoV-2 activity of Huangqi. The inconsistency may be due to the difference of TCM preparations and cell lines. We used normal bronchial epithelium BEAS2B rather than Vero E6 derived from African green monkey kidney epithelium to mimic respiratory infection close to real clinical situation. In addition, we used EAM B, a more purified preparation of Huangqi instead of crude water extract, to test the anti-SARS-CoV-2 activity.

In conclusion, a novel TCM candidate could be characterized through in silico and in vitro pathway analysis and anti-viral activity assay. Bioinformatics-based drug screening may gain promising prospects not only in predicting and repurposing old drugs, but also in unveiling innovative potential for future study of TCM

### Example 6

Huangqi on the Binding of SARS-CoV2-Spike with ACE2 and Syncytia Formation.

The host cell invasion of SARS-CoV-2 starts with the binding of viral spike protein to the human ACE2 receptor enabled by the cleavage of the receptor-binding domain (RBD) region on S1 from the spike protein during viral binding and entry procedure. This process of accessing host cells, dependent on the interaction between the complex sugar molecules (glycans) on the surface of viruses and host cells via glycoproteins, is required for SARS-CoV-2 replication. Glycans found on the spike protein are only marginally involved in the binding of the virus to human cells; however, they are vital in the virus' fusion with the host cell and cell entry.

EGFP/spike-positive BHK-21 cells were incubated on a single-layer of Calu-3 cells in the absence or presence of Huangqi (1000 μg/ml of EAM B or EAM A) at 4° C. for 1 h. After washed out unbound cells by PBS, five fields were randomly captured to acquire initial fluorescence images representing for binding efficiency. The cells were then incubated at 37° C. for another 4 h. The fluorescence images of the cells acquired at 4 h represented for fusion efficiency. The binding efficiency of SARS-CoV2-spike to ACE2 (gray bars) and the formation of syncytium indicating fusion efficiency (white bars) were quantified in the cells treated with extract of 1000 μg/ml of EAM B or EAM A).

Result

Although 1000 μg/ml EAM B or EAM A could reduce syncytia formation without decreasing the binding of the EGFP-positive cells, allowing 80% and 70% of the level of cell fusion of the control groups, respectively, the reductions were not statistically significant (FIGS. 4A and 4B).

Since Huangqi contains polysaccharides, we hypothesized that Huangqi might compete with the glycans on the spike proteins; this hypothesis could explain Huangqi's role in fusion blocking despite its statistically insignificant data (FIGS. 4A and 4B). Therefore, it is essential to investigate further mechanisms of those treatments on spike glycans and whether it is feasible to bind to glycans on ACE2.

### Example 7

Sulforhodamine B Colorimetric (SRB) Assay

Cells were seeded (2,000 cells per well) for 16-20 h, and then treated with different concentrations of the drug for 24 h. The medium was discarded and cells were fixed by cold 10% trichloroacetic acid (w/v) (SIGMA) at 4° C. for 1 h. After fixation, plates were washed twice with water. Cells were stained with 100 μl/well of 0.1% (w/v, in 1% acetic acid) SRB solution at RT for 1 h, and then washed twice with 1% acetic acid (AVENTOR). After air-drying, 100 μl of 20 mM Tris-base was added to each well and was read at OD 540 nm.

The Cell-Cell Fusion Assay

Calu-3 were first seeded in 12-well plate (1×10⁶ per well) to form a single-layer of cell films as target cells. BHK-21 cells (4×10⁵ per well) were transfected with both EGFP and Spike plasmids (the original Wuhan strain) in a ratio of 1:5 for 24 h, harvested using Cell Dissociation Buffer (Gibco) and resuspended in serum free DMEM (Gibco). EGFP/Spike-coexpressing BHK-21 cells that were used as donor cells were incubated on a single-layer of Calu-3 cell film for cell-cell contact at 4° C. for 45 minutes prior to PBS washing and growth medium replacing to removing unbound cells. After 4° C. incubation, the temperature was switched to 37° C. and cells were incubated for additional 4 hours. Five fields of GFP-expressing cell images were randomly acquired by an inverted fluorescence microscope (Olympus IX70) at time points, 0 and 4 hours, respectively. To quantify binding efficiency of GFP-positive BHK-21 cells with Calu-3 cells in both control and TCM-treated groups, the initial numbers of GFP-positive BHK-21 cells attached with Calu-3 cells were counted. The number of GFP cells in control groups was defined as 100% of the binding efficiency. The effects of TCM on the bind efficiency were determined by the percentage of the binding efficiency normalized by control. To quantify the formation of syncytium form, the extension area of GFP-expressing cell images was quantified by ImageJ software. The fold change of GFP area in control groups from 0 to 4 h was delimited to 100% of the fusion efficiency. The effects of TCM on the syncytium formation were calculated according the following equation:

$$Normalized percentage ( \% ) = \frac{the fold change of GFP area in TCM - treated group}{the fold change of GFP area in control group} \times 100$$

Result

Suppression of the Binding of SARS-CoV-2 Spike Protein with ACE2 and the Formation of Syncytium Implicated a Potential Therapeutic Role of EAM C.

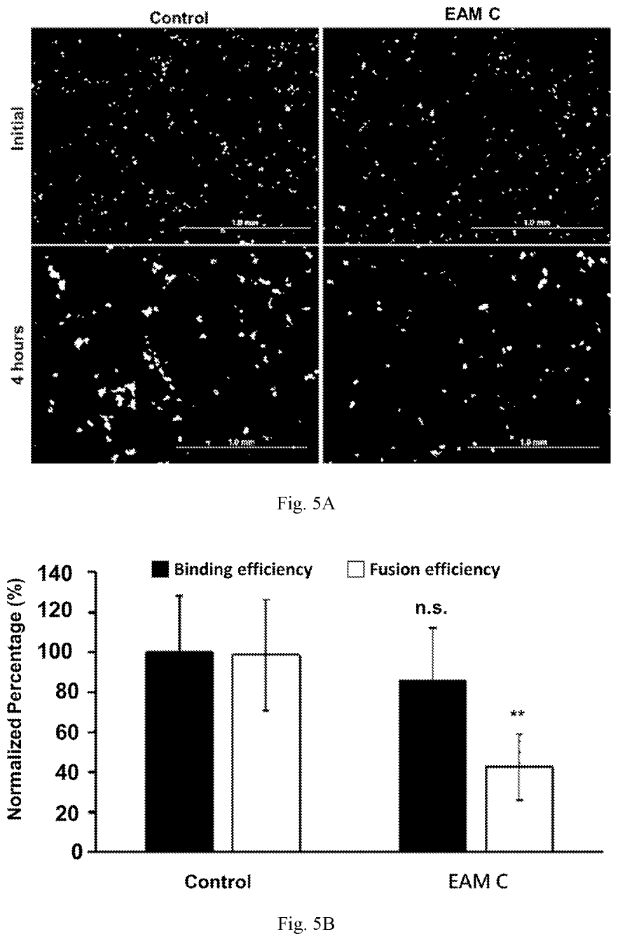

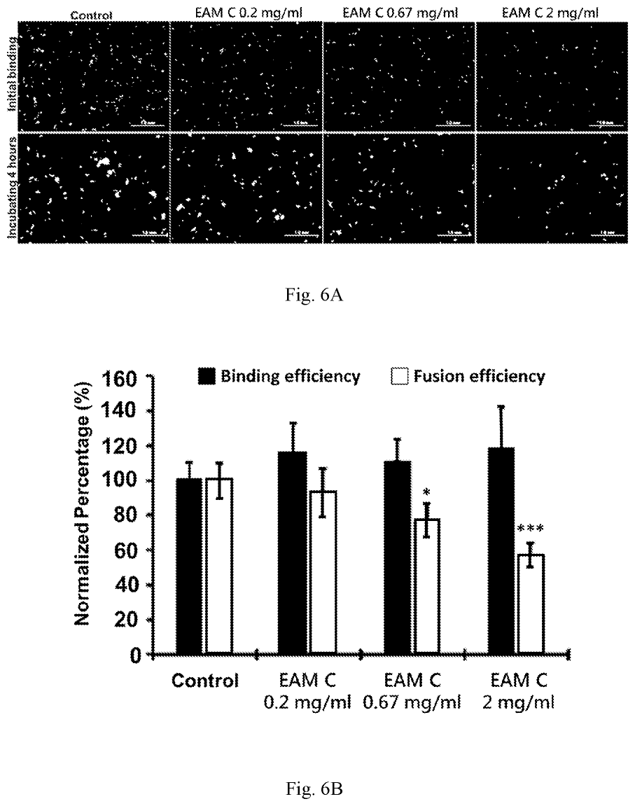

Receptor-dependent syncytia formation is triggered by SARS-CoV-2 spike (S) protein on the cell membrane. Thus, we evaluated the anti-SARS-CoV-2 activity of EAM C by measuring the binding efficiency between spike protein (BHK-21 cells expressing SARS-CoV-2 S protein and EGFP) and its corresponding receptor protein (Calu-3 cells expressing endogenous hACE2 receptor). The binding of the BHK-21 cells to the Calu-3 cells indicated the binding of the SARS-CoV-2 S protein with the ACE2 receptor. Also, the formation of syncytium resulting from the membrane fusion between BHK-21 and Calu-3 cells was measured. In FIG. 5A-6B, we observed a 20% reduction of the number of EGFP-positive cells binding in EAM C 1 mg/ml, 0.2 mg/ml, 0.67 mg/ml or 2 mg/ml treatment compared to the control, although the decrease was not statistically significant (FIGS. 5A and 6A, upper panel; FIGS. 5B and 6B, gray bars). In contrast, the treatment of EAM C significantly reduced the formation of syncytium, compared to the control group (FIGS. 5A and 6A, bottom panel). Quantitative analysis revealed that EAM C could inhibit 60% of cell-cell fusion (FIG. 5B, white bars). These results suggested that EAM C could potentially be used as an anti-SARS-CoV-2 agent by blocking SARS-CoV-2 S protein-mediated membrane fusion.

### Example 8

Reduction of Binding Efficacy of Wild-Type, Alpha, Delta, Gamma, and Delta of the Trimeric Spike Protein Binding to ACE2

Enzyme-linked immunosorbent assay (ELISA) is used to evaluate the ability of EAM C to intervene the binding of trimeric SARS-COV-2 Spike protein wild type (Whuan strain) or variants (alpha, delta, gamma, and Delta) to biotinylated human ACE2 recombinant protein. Experimental procedures are described as follow. Firstly, 100 μl of spike protein (500 ng/ml; cat. GTX135972-pro, GeneTex, Taipei, Taiwan) diluted in coating buffer, consisting of sodium carbonate (15 mM), sodium hydrogen carbonate (35 mM), pH 9.6, was added into each well of 96-well. Plate was sealed and kept for overnight at 4° C. Secondly, coated plate was washed three times with washing buffer, consisting of PBS with 0.05% (v/v) Tween-20 (pH 7.4). Then, plate was further blocking with 300 μl of blocking buffer which is made of washing buffer containing 0.5% (w/v) bovine serum albumin (BSA) for 1.5 h at 37° C. After washed three times, 100 μl of test agent or inhibitor (10 μg/ml; cat. GTX635791, GeneTex, Taipei, Taiwan) in dilution buffer was added and incubated for 1 hr at 37° C. 100 μl of biotinylated human ACE2 protein (125 ng/ml; cat. AC2-H82E6-25 ug; ACROBiosystems, OX, UK) was further added into each well and incubated for 1 hr at 37° C. Afterwards, plate was washed three times with wash buffer. Finally, 100 μl of Streptavidin-HRP conjugates (100 ng/ml; cat. GTX30949, GeneTex, Taipei, Taiwan) in dilution buffer was added and incubated for 1 hr at 37° C. At the end, washed plate was incubated with 200 μl of TMB substrate per well for 1 hr at 37° C. under light protection. 50 μl of stop solution was used to terminate the reaction and the absorbance is detected at 450 nm by using a microplate reader (Cytation 5, BioTek, Vermont, USA).

Result

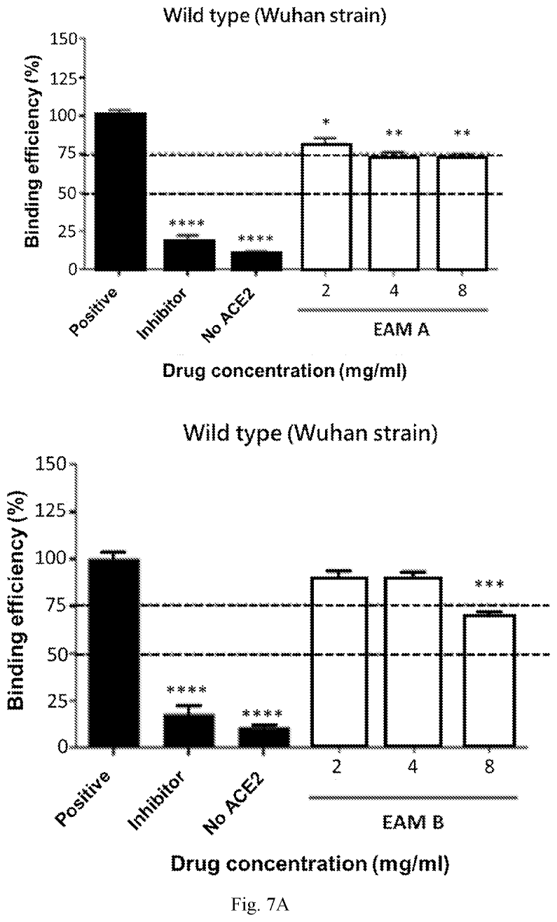

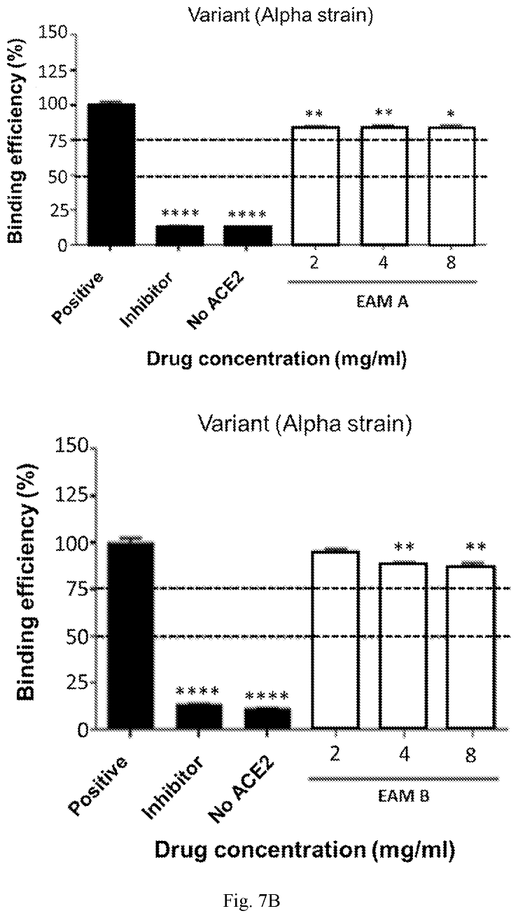

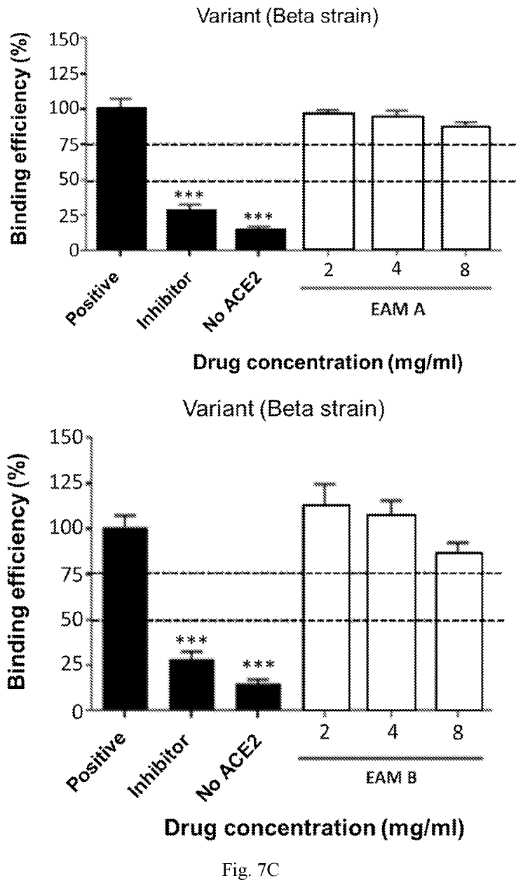

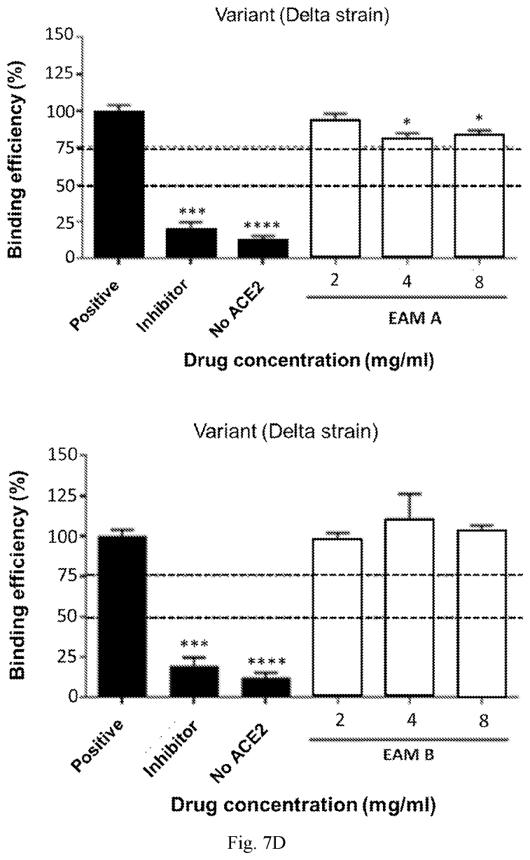

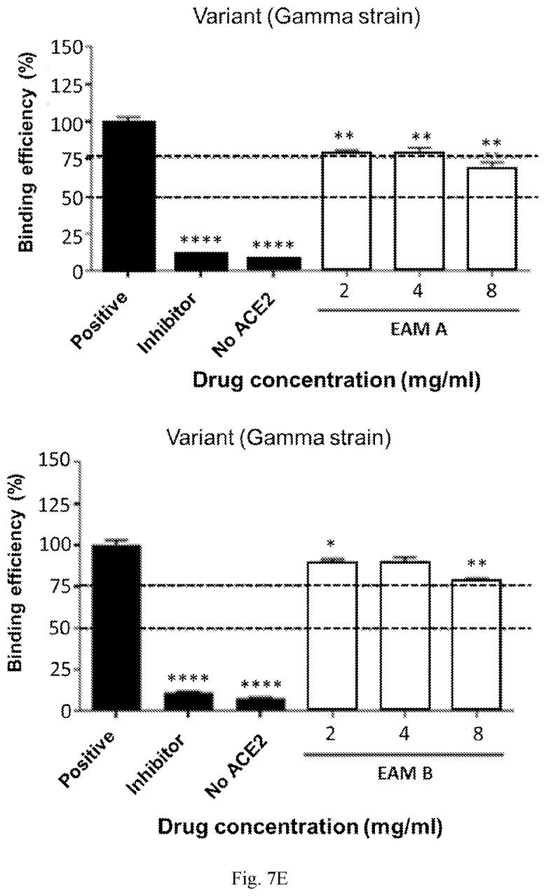

EAM A showed better effect with the reduction of binding efficacy of wild-type, alpha, delta, and gamma of the trimeric spike protein binding to ACE2 reaching statistical analysis. The results showed that Huangqi may inhibit the binding and fusion stage of SARS-CoV-2 infection. EAM B showed better effect with the reduction of binding efficacy of wild-type, alpha, delta, and gamma of the trimeric spike protein binding to ACE2 reaching statistical analysis. The results showed that Huangqi may inhibit the binding and fusion stage of SARS-CoV-2 infection. (FIG. 7A-7E)

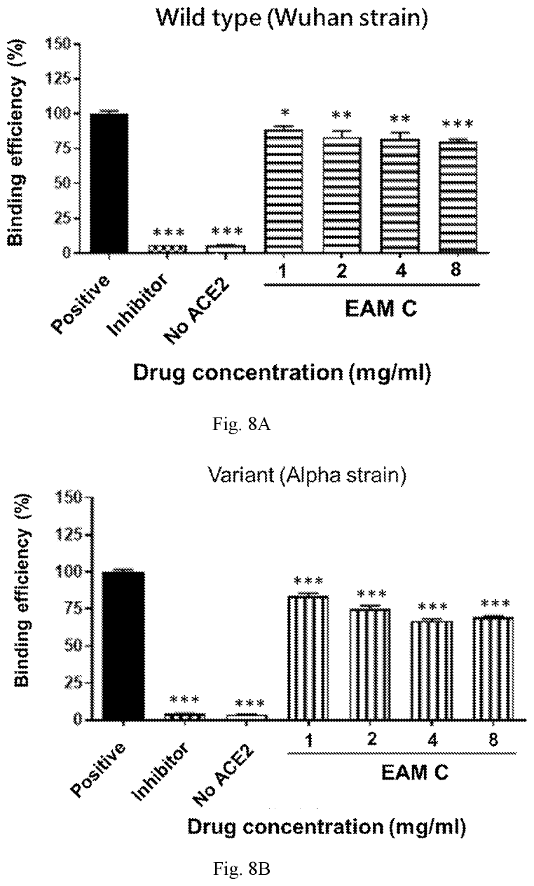

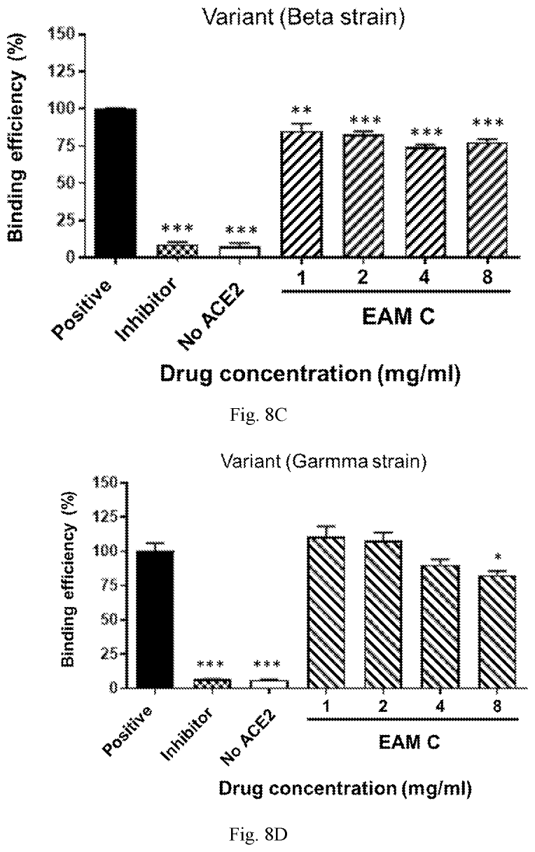

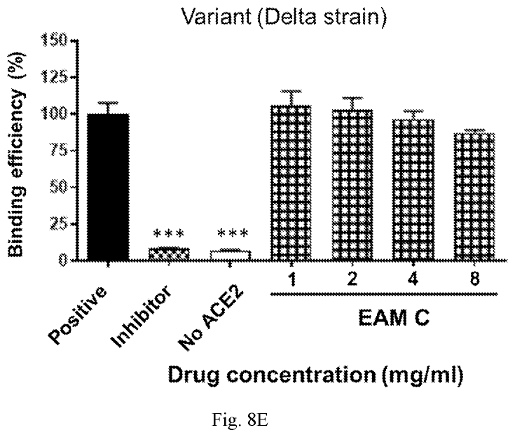

By employing ELISA-based trimeric spike protein binding assay, potential inhibitory effects of EAM C on the current circulated variants were compared. As shown in FIG. 8, the presence of EAM C at concentrations between 1 to 8 mg/ml significantly reduced binding efficiency of the trimeric spike protein from wild-type, alpha, and beta variants (FIG. 8A-8C). Among those three types, EAM C appeared to be more effective when confronting spike proteins from alpha and beta strain. In contrast, EAM C only provided a modest effect on gamma variant (FIG. 8D) and had no effect on delta variant (FIG. 8E).

All examples provided herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority or inferiority of the invention. Although one or more embodiments of the present invention have been described in detail, it should be understood that the various changes, substitutions, and alterations could be made hereto without departing from the spirit and scope of the invention.

It is intended that the specification and examples be considered as examples only, with a true scope and spirit of the invention being indicated by the following claims.

What is claimed is:

1. A method for preventing the SARS-Cov-2 infection in a subject, comprising administering to the subject an extract of Astragalus membranaceus, wherein the extract can suppress the binding of the SARS-CoV-2 viral spike protein to the human ACE2 receptor.

2. The method of claim 1, wherein the extract of Astragalus membranaceus is selected from the group of EAM C, EAM A and EAM B, wherein the EAM C is the extract containing 85%-100% of total sugar content, wherein the EAM A is the extract containing 55%-80% of total sugar content, wherein the EAM B is the extract containing 25%-50% of total sugar content.

3. A method for treating or relieving the SARS-Cov-2 infection in a subject, comprising administering to the subject an extract of Astragalus membranaceus, wherein the extract targeting a miR-146a, a let-7a or a miR-148b, elevating the miR-146a, the let-7a or the miR-148b expression to perform its normal cellular function.

4. The method of claim 3, wherein the extract of Astragalus membranaceus is selected from the group of EAM C, EAM A and EAM B, wherein the EAM C is the extract containing 85%-100% of total sugar content, wherein the EAM A is the extract containing 55%-80% of total sugar content, wherein the EAM B is the extract containing 25%-50% of total sugar content.

5. The method of claim 3, the extract of Astragalus membranaceus can reduce the SARS-CoV2 M^{pro} activity IC₅₀ concentration.

6. A method for preventing the cytokine storm caused by the SARS-CoV-2 infection in a subject, comprising administering to the subject an extract of Astragalus membranaceus, wherein the extract targeting a miR-146a, a let-7a or a miR-148b, elevating the miR-146a, the let-7a and the miR-148b expression to perform its normal cellular function.

7. The method of claim 6, wherein the extract of Astragalus membranaceus is selected from the group of EAM C, EAM A and EAM B, wherein the EAM C is the extract containing 85%-100% of total sugar content, wherein the EAM A is the extract containing 55%-80% of total sugar content, wherein the EAM B is the extract containing 25%-50% of total sugar content.

8. The method of claim 6, wherein the extract of Astragalus membranaceus can reduce the IL-6, the IL-10 or the TNF-α expression.

9. A method for preventing the cytokine storm in a subject, comprising administering to the subject an extract of Astragalus membranaceus, wherein the extract targeting a miR-146a, a let-7a or a miR-148b, elevating the miR-146a, the let-7a or the miR-148b expression to perform its normal cellular function.

10. The method of claim 9, wherein the extract of Astragalus membranaceus is selected from the group of EAM C, EAM A and EAM B, wherein the EAM C is the extract containing 85%-100% of total sugar content, wherein the EAM A is the extract containing 55%-80% of total sugar content, wherein the EAM B is the extract containing 25%-50% of total sugar content.

11. The method of claim 9, wherein the extract of Astragalus membranaceus can reduce the IL-6, the IL-10 or the TNF-α expression.
